(19) United States
(12) Patent Application Publication
(10) Pub. No.: US 2026/0234094 A1
(43) Pub. Date: Aug. 13, 2026

(54) PROCESS FOR THE PREPARATION OF HYDROXYPHENYLCARBOXYLIC ACID ESTERS USING CATALYTIC TRANSESTERIFICATION

(71) Applicant: BASF SE, Ludwigshafen am Rhein (DE)

(72) Inventors: Sina WITZEL, Ludwigshafen am Rhein (DE); Martin SUKOPP, Ludwigshafen am Rhein (DE); Christian REIN, Ludwigshafen am Rhein (DE); Tiziano NOCENTINI, Muttenz (CH)

(21) Appl. No.: 19/156,608

(22) PCT Filed: Feb. 14, 2024
(86) PCT No.: PCT/EP2024/053703

§ 371 Date: Aug. 14, 2025

(30) Foreign Application Priority Data
Feb. 17, 2023 (EP) 23157396.5

(51) Int. Cl. C07C 67/02; B01J 27/232

(52) U.S. Cl. CPC C07C 67/02; B01J 27/232

(57) ABSTRACT

A process for the preparation of hydroxyphenylcarboxylic acid esters comprising a catalytic transesterification step using alkali metal carbonates or alkali metal bicarbonates as catalysts. The transesterification process, wherein (a) a vessel is charged with a substituted phenol comprising a carboxylic methoxy ester moiety, which moiety is linked directly—or indirectly via an alkyl chain CₘH₂ₘ—to the phenolic ring, and a fatty alcohol, (b) the mixture of the two components is heated above the melting point of the two components and vacuum is applied, and an alkali metal carbonate or an alkali metal bicarbonate is already present at step (b) or is added to the liquid mixture at step (c), and the transesterification reaction is allowed to happen. The use of alkali metal carbonates or alkali metal bicarbonate as catalysts leads to a safe, sustainable, environmentally friendly process, which avoids toxic side products and does not require extensive conditioning.

### BACKGROUND OF THE INVENTION

Carboxylic esters can in general be prepared by reacting carboxylic acids with alcohols. This reaction can be carried out autocatalytically or catalytically, for example by means of Brønsted or Lewis acids. In many cases, metal compounds are used as catalysts, such as the alkoxides, carboxylates and chelate compounds of titanium, zirconium, tin, zinc and aluminum.

Even though the catalytic properties of these metallic catalysts are satisfactory, the removal of the catalyst residues from the esterification products presents difficulties. For purification, the crude esters are generally first admixed with alkali metal hydroxides to neutralize unconverted or incompletely converted acid (partial esters), and the free alcohols are removed by steam distillation. After brief vacuum distillation to dry the product, the catalyst residues are then removed by filtration. Since the catalyst residues are generally of slimy, gel-like consistency, filtration is usually possible only with the aid of filtration aids, for example activated carbon, wood flour, cellulose or celite. Nevertheless, such a filtration under these circumstances is still associated with serious disadvantages: long filtration times are required, and the yield of ester is reduced because large amounts of product are retained in the filtercake.

Carboxylic esters may also be prepared by a transesterification reaction. Hydroxyphenylcarboxylates of formula (I) as shown further below for instance, which act as antioxidants in many fields of the chemical industry, have been known to be prepared by different methods including as well by a number of transesterification processes (e.g. U.S. Pat. Nos. 3,330,859; 3,944,594; 4,085,132; 4,228,297; 4,536,593; 4,594,444; 4,618,700; 4,716,244). But also these processes were not entirely satisfactory. Thus, for example, the titanium compounds used as catalysts were often difficult to separate from the reaction mass. Once deployed, they often had to be destroyed by troublesome procedures and arrangements had to be made for disposal of the filtration residues.

In particular, catalyst residues in the product could result in unwanted oxidation reactions which discolor the products.

Hence, in order to improve those processes, lot of efforts had been made in the past.

For example, aluminium alcoholates, which had already been known as esterification and transesterification catalysts and which had been used for the preparation of ally β-phenylpropionates by transesterification (FR-A-1 490 341) for the perfume industry, have also been suggested for use in those processes.

For example, U.S. Pat. No. 5,481,023 describes the use of aluminium alcoholates as catalysts to obtain hydroxycarboxylates as cleanly, in good yield, without separation and oxidation problems, and with the aid of environmentally acceptable auxiliaries.

This use may resolve some of the problems mentioned above, however, others still remain.

For instance, as mentioned above, residues of catalyst may not only cause optical or aesthetic disadvantages, like said coloration or discoloration of the product, they may also be considered critical with regard to environmental issues, toxicity issues or food safety.

In case of the use aluminium as a catalyst for the preparation of compounds or materials, which are later used for the manufacture food containers, the latter are required to be free of aluminium residues derived from the application of aluminium as a catalyst.

In this regard, authorities have adopted regulations on the safety of aluminium from dietary intake, which establishes a tolerable weekly intake of 1 mg aluminium per kg body weight per week. Applying the conventional exposure assumptions for food contact materials, the migration limit would have to be set at 8.6 mg/kg food.

However, the European authorities for instance deemed it appropriate to limit the contribution from exposure by food contact materials to the overall exposure by applying an allocation factor of 10% to the conventionally derived migration limit. Therefore, a migration limit for aluminium of 1 mg/kg food is considered appropriate for food contact materials. The leads to the regulation that plastic materials and articles with food contact shall not release aluminium in quantities exceeding the specific migration limits of aluminium of 1 mg/kg food or food simulant (COMMISSION REGULATION (EU) 2016/1416 of 24 Aug. 2016 amending and correcting Regulation (EU) No 10/2011 on plastic materials and articles intended to come into contact with food).

Hence, in view of regulations becoming stricter for consumer safety and environmental protection, there is a strong need for novel, improved and sustainable processes for preparing compounds, which will later be applied in processes for preparing food containers or other material having food contact.

Thus, in view of the above said, making efforts for continuous improvement, it is therefore an object of the present invention to develop processes for transesterification which still meets the requirements with regard to the technical performance on the one side, but which—in addition—provide consumer safety and environmental issues.

Surprisingly, it has now been found that, by using alkali metal bicarbonates or alkali metal carbonate, wherein the alkali metal is either sodium or potassium, as catalysts for these transesterification processes, it is possible to obtain the hydroxycarboxylates and other hydroxycarbonyl-derivatives described hereinafter cleanly, in good yield, without separation and oxidation problems and in an environmentally friendly and sustainable way. The hydroxycarboxylates and other hydroxycarbonyl-derivatives are not only obtained by an improved process (e.g. such as by simplified filtration, use of easy accessible, non toxic catalysts), the obtained product itself is characterized by a lower transmission rate, a significant lower rate of side products (if any) and thereby by a superior quality, making it suitable for consumer-safely and environmentally harmless applications.

Although alkali metal carbonates or alkali metal hydrogen carbonates, the latter also commonly and hereinafter called alkali metal bicarbonates, have appeared in transesterification processes in the prior art, the claimed sodium or potassium salts were never used as a sole catalyst for the transesterification process according to the present invention per se.

CN112048030 relates to a polyethylene grafted hindered phenol antioxidant, and a preparation method thereof, whereby the method for preparing the polyethylene grafted hindered phenol antioxidant comprises the steps of mixing a polyethylene containing a hydroxyl group at side chain with a propionate derivative, which is subjected to transesterification reaction under the action of a catalyst. Mentioned catalyst are selected from tin compounds, titanium compounds, alkali metal compounds, alkaline earth metal compounds, whereby (n-butyl)tin oxide, (dibutyl)tin dilaurate, lithium amide and sodium amide are preferred, and the exemplified catalyst show dibutyl maleate, calcium hydroxide and tin t-butoxide.

JP06107596 relates to a preparation method of tetrakis[3-(3,5-dialkyl-4-hydroxyphenyl)propionyloxyalkyl]methanes via catalyzed transesterification, wherein the basic catalyst may be selected from a long list of presented catalysts. Examples of used catalyst disclose lithium methoxide, calcium oxide and tin t-butoxide.

EP0608089 relates to alkoxyalkylene glycol esters of substituted phenylpropionic acids and to their use as antioxidant stabilizers for organic materials subject to oxidative deterioration, such as synthetic polymers and resins. The esters in EP0608089 can be prepared by various esterification methods, including the transesterification between the alkylene glycol monoether and a substituted phenylpropionic acid ester. The reaction may be performed in the presence of a transesterification catalyst which include, for example, alkali metals, alkali metal amides, alkali metal alkoxides, alkali metal hydroxides, titanium (IV) alkoxides and metal oxide salts, wherein the alkali metal alkoxides are explicitly preferred. And although alkali metal carbonates and alkali metal bicarbonates had been mentioned as well in this list, they are not found used for this purpose in the description, but merely as washing agent after completion of the transesterification reaction. For example, the obtained ester is isolated by means of conventional techniques, whereby the reaction mixture is washed and neutralised with a dilute mineral acid (e.g. dilute hydrochloric or sulfuric acid) or with an aqueous alkali solution (e.g. aqueous sodium bicarbonate), like e.g. in example 1, washed with 5% aqueous sodium bicarbonate solution and water.

Similar conditions are to be found described for JP08092165 from the same applicant, wherein a new wax-like antioxidant having low pour point and excellent antioxidizing effect is described.

The antioxidant contains esters obtained by reacting a substituted phenyl alkanoic acid with an alkylene oxide adduct of a polyhydric alcohol as an active ingredient, for which alkali metal carbonates and alkali metal bicarbonates are mentioned as well in a long list of optional transesterification catalyst. However, they are not found used for this purpose in the description, but merely as washing agent after completion of the transesterification reaction. In example 4, after the transesterification reaction has taken place, the reaction solution is cooled to room temperature, and 0.1 N-hydrochloric acid, water and 4% sodium bicarbonate aqueous solution are used for washing.

And EP3067343, which relates in particular to new antioxidants and polyurethane foam, mentions a process comprising a transesterification between the alkylene glycol monoether and the substituted phenylpropionic acid ester and mentions further that this implementation may be carried out in the presence of a transesterification catalyst, which may be selected for example from alkali metals, alkali metal hydrides, alkali metal amides, alkali metal alkoxides, alkali metal hydroxides, alkali metal carbonates, alkali metal bicarbonates, carboxylic acid salts of alkali and alkaline earth metals (eg acetates or formates), aluminum alcoholates and phenates, titanium (IV), metal oxides or organic acids and mineral acids and sulfonic acids. In particular sulfuric acid as the mineral acid and p-toluenesulfonic acid as the sulfonic acid are preferred. The alkali metal alkoxides are also pointed out to be preferred.

Hence, in all these three cases of the prior art, EP0608089, EP3067343 and JP08092165, the transesterification catalyst used were either p-toluenesulfonic acid or metal oxides such as sodium methoxide, sodium acetoxide or titanium (IV) tetrabutoxide. None of the prior art discussed any of the aforementioned problems and challenges to be resolved, nor was it suggested to pick an alkali metal carbonate or bicarbonate even randomly, not to mention to address these kinds of problems. In fact, the alkali metal bicarbonates were added to the mixture of reactant only after the transesterification step had taken place, and for a completely different purpose, namely as washing agents.

As can be seen from the prior art cited herein above, transesterification reaction steps are known in preparation methods in the field of antioxidants, and long lists of optional transesterification catalysts are mentioned. However, even if alkali metal (bi)carbonates are found to be mentioned in this prior art, they are mentioned to be used for a variety of functions, such as halide scavengers or as washing agents, whereby being exemplified only in the latter, but not being exemplified as being a transesterification catalyst, not to mention any of their abilities or advantages with regard to the problems to be solved according to the present invention.

CN 104447333 discloses a method for preparing isooctyl 3,5-di-tert-butyl-4-hydroxyphenyl propionate by reaction of methyl 3,5-di-tert-butyl-4-hydroxyphenyl propionate with isooctyl alcohol at 140-170° C. under vacuum at 0.07-0.08 MPa for 4-8 hours in the presence of lithium carbonate. The selection of different counterions show significant variable and non-predictable effects. Specific interactions between the counterions and other present reactants may lead to surprising and unexpected results. The use of Li₂CO₃ may therefore not be regarded as leading to the specific use of potassium or sodium.

SU1014213 describes a method of producing pentaerythryl-tetrakis-[β-(3,5-di-tert-butyl-4-hydroxyphenyl)propionatel] by transesterification of methyl (3,5-di-tert-butyl-4-hydroxyphenyl)-propionic acid methyl ester with pentaerythritol. It thereby discusses the methods of the prior art by then (1981), wherein the transesterification took place in the presence of catalysts such as alkali metals, their alcoholate hydrides, and especially lithium hydroxide. The disadvantages of this method with regard to the use of this explosive catalyst are addressed. SU1014213 discusses as well the disadvantages of another method using sodium methylate as a catalyst, which is sensitive to the presence of moisture in the initial reagents and requiring special conditions for storage and dosage.

The advantages of the transesterification method suggested in SU1014213 are described by the use of easily available and cheap catalysts, which address the technical handling of the transesterification method, such as shortening the reaction time, avoiding catalyst neutralization stages and the exclusion of wastewater formation, but which certainly do not address the problems of modern times with regard to environmental sustainability and consumer safety.

This can especially be recognized, that the goal presented in SU1014213 is achieved by the use of a catalyst, which is consisting of a mixture of potassium carbonate, sodium carbonate and phenol, whereas the weight ratio is of 1:1.5-2:2.5-3.

The first essential difference is that the present invention uses a single compound as catalyst, which selected from alkali metal carbonates or alkali metal bicarbonates for the transesterification reaction step, and not a mixture.

Especially, the present invention does not require the presence of an aromatic compound such as phenol to be used as catalyst. Phenol is a protoplasmic poison with myriad effects. Its dual hydrophilic and lipophilic properties allow it to easily break through cellular membranes, denaturing proteins along the way (explaining its widely use as disinfectant), and ultimately leading to cell death and necrosis, as well as caustic effects resulting in coagulation necrosis.

Obviously, it should be avoided in any matter with regard to food applications.

Furthermore, the weight ratio of potassium carbonate to sodium carbonate to phenol of 1:1.5-2:2.5-3 shows that the phenol has considerable ratio of around 1:1 in comparison to the two combined alkali metal carbonates (1+1.5 up to 1+2) leading from 2.5-3 to 2.5-3, thereby demonstrating that it is required in a not neglectable amount.

Apart from the consistency and toxicity of the catalyst mixtures, further technical parameters and features of the present invention differ widely from the ones described in SU1014213. Although the process in SU1014213 describes a transesterification reaction step, wherein alkali metal carbonates are used, said process differs in many technical features and parameters from the transesterification reaction step of the present invention.

SU1014213 provides a method for obtaining pentaeryl-tetrakis-(5-(3,5-ditertbutyl-4-hydroxyphenyl)-propionate, according to which the starting methyl ester -(3,5-ditertbutyl1-4-hydroxyphenyl)-propionic acid and pentaerythritol are heated to 135-150° C., and a residual pressure of 20-100 mm Hg is applied for 9 hours in the presence of a catalyst used in the amount of 6-10% of the weight of the starting methyl ester.

The required concentration of the catalyst according to the present invention is considerably lower, as it is preferably added to the mixture of the starting methyl ester (compound of formula (II)) AND compound of formula (III) in concentration range of less than 10 wt %, preferably less than 5 wt %.

Further, as can be seen in the examples provided hereinafter, the transesterification reaction step of the present inventions is allowed to take place for 1 to 8 hours, preferably from 4 to 6 hours, wherein the best results, meaning the best conversion rate is to be expected, whereas SU1014213 describes the transesterification reaction to require 9 hours.

And finally, the yield of the transesterification reaction obtained according to the process of SU1014213 is 78-83% (expressed in GC area %), whereas the conversation rate according to the present inventions reaches at least 82.62% to 89.69% for sodium carbonate and up to 93.20% for the potassium bicarbonates, thereby showing a much higher efficacy compared to SU1014213

In Ter Haar Ruud et al. in Carbohydrate Research (vol. 346, no. 8, June 2011, p. 1005-1012) the synthesis of hydroxy-aryl esters of glycosidic polyols and non-reducing oligosaccharides is discussed. In order to enable the enzymatic coupling of saccharides to proteins, several di- and trisaccharides were hydroxy-arylated using anhydrous transesterification with methyl 3-(4-hydroxyphenyl)propionate, catalysed by potassium carbonate. Although K₂CO₃ is used as a catalyst, the reaction mixture is strictly anhydrous and requires DMSO (Dimethyl Sulfoxide) as activating solvent, which is a polar aprotic solvent. The present invention, however, does not require the presence of a solvent at all, and the use of an activating solvent like DMSO would lead to undesired side effects. In addition, the publication refers strictly to sugar chemistry, which is a special class in the chemical organic field. Although sugar molecules consist solely of the organic elements carbon, oxygen and hydrogen, the unique aspect of sugar structures, wherein the carbon, hydrogen, and oxygen atoms are in a very specific arrangement, forming ring-like structures provides them with individual chemical properties, whereby they behave differently and are not comparable with the chemical behaviour of non-sugar organic molecules. Sugar molecules are chemically highly sensitive, especially with regard to heating. Hence, a transesterification process, which requires the presence of an activating solvent such as DMSO in the field of sugar chemistry cannot be applied analogously in a transesterification process in the non-sugar chemical field.

### SUMMARY OF THE INVENTION

The invention therefore relates to the use of alkali metal carbonates or alkali metal bicarbonates as catalysts for the preparation of compounds of formula (I) by reacting compounds of formula (II) with compounds of formula (III) as shown further below.

The inventive improvement of the process is the use of alkali metal carbonates or alkali metal bicarbonate as catalysts, which leads to a safe, sustainable, environmentally friendly process, which avoids toxic side products and does not require extensive conditioning.

The object was surprisingly achieved by a transesterification process, wherein
a) a vessel is charged with a substituted phenol comprising a carboxylic methoxy ester moiety, which moiety is linked directly—or indirectly via an alkyl chain CₘH₂ₘ—to the phenolic ring, and a fatty alcohol,
b) the mixture of the two components is heated above the melting point of the two components, and vacuum is applied;
c) an alkali metal carbonate or an alkali metal bicarbonate is added to the liquid mixture and the transesterification reaction is allowed to happen.

The process is especially characterized of being carried out in a water-free, non-aqueous environment. Water-free in the context of the present inventions means, that no addition of water is required. On the contrary, the addition of water would preclude the desired transesterification reaction, as the addition of water would lead to hydrolytic effects.

However, the presence of some residual water within the compounds may be tolerated, such as crystal water of sodium bicarbonate.

Nevertheless, the starting compounds are preferably, but not necessarily, dried before the transesterification reaction. In general, the heating-up of the vessel and pulling vacuum is already sufficient to remove the residual water from the starting compounds.

The transesterification reaction is started as soon as the alkali metal carbonate or an alkali metal bicarbonate is added to the mixture of the two components and the mixture is heated. The process can be conducted in two alternative ways: Either continuously, in which case the individual steps are performed in continuously operated apparatuses connected in series, or alternatively, the process can be performed batchwise.

The transfer reaction type concept can be used as well not only with fatty alcohols, but as well with other linear, branched or cyclic alkyl derivatives having 1, 2, 3 or 4 functional group selected from HO, H₂N or HS.

### DETAILED DESCRIPTION OF THE INVENTION

The process according to the present invention is a non-aqueous process for the preparation of a compound of formula (I)

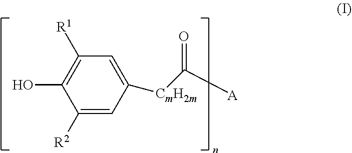

wherein
   R₁ and R₂ are independently of one another hydrogen or a linear or branched C₁-C₈-alkyl,
   m is either 0 or an integer selected from 1, 2 or 3,
   n is an integer selected from 1, 2, 3 or 4,
   and
   A is
      if n is 1, OR₃, and R₃ is C₄-C₂₀-alkyl or C₅-C₁₂ cycloalkyl; or
      if n is 2, has the formula —O—CₓH₂ₓ—O—, O—CₓH₂ₓ—S—CₓH₂ₓ—O, —NH—CₓH₂ₓ—NH—or —O—(CH₂CH₂O)ₐCH₂CH₂O—,
         wherein
         x is an integer selected from 2 to 8, and
         a is an integer selected from 1 to 12; or
      if n is 3, has the formula H₃C—CₓH₂ₓ—C(C_{y}H_{2y}O—)₃, —O—CₓH₂ₓ—CH₂(O—)—C_{y}H_{2y}O—,
         wherein
         x,y are independently from one another an integer selected from 1 to 8; or
      if n is 4, has the formula —C(—CH₂—O—)₄
   by a transesterification reaction, wherein a compound of formula (II)

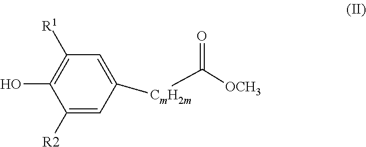

Is reacted with a compound of formula (III)

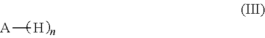

And wherein the process step of the transesterification reaction between compound of formula (II) and compound of formula (III) is characterized by being started by the addition of catalyst consisting of an alkali metal bicarbonate OR an alkali metal carbonate selected from NaHCO₃, Na₂CO₃, KHCO₃ and K₂CO₃, and which is present in its undissolved solid form.

In one embodiment of the process according to the present invention, the integer of m in formula (I) or (II) is 2.

In one embodiment of the process according to the present invention, R₁ and R₂ are both selected independently from one another from a linear or branched C₁-C₄ alkyl.

In one embodiment of the process according to the present invention, R₁ and R₂ are both the same linear or branched C₁-C₄ alkyl, preferably R₁ and R₂ are both tert-butyl.

In one embodiment of the process according to the present invention, R₁ and R₂ are both tert-butyl.

In one embodiment of the process according to the present invention, the integer of n in formula (I) or (II) is 1.

In one embodiment of the process according to the present invention, the integer of n in formula (I) or (II) is 1, and R₃ of OR₃ in A is C₈-C₁₈ alkyl.

In one embodiment of the process according to the present invention, the integer of n in formula (I) or (II) is 1, and R₃ of OR₃ in A is C₈-alkyl.

In one embodiment of the process according to the present invention, a compound of formula (I.1) is obtained:

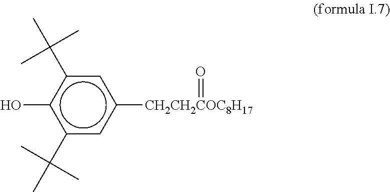

In one embodiment of the process according to the present invention, the integer of n in formula (I) or (II) is 1, and R₃ of OR₃ in A is C₁₈-alkyl.

In one embodiment of the process according to the present invention, a compound of formula (I.2) is obtained:

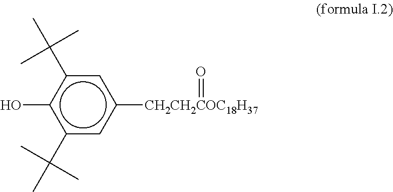

In one embodiment of the process according to the present invention, the integer of n in formula (I) or (II) is 2.

In one embodiment of the process according to the present invention, the integer of n in formula (I) or (II) is 2, and A of formula (I) or (III) has the formula —O—CₓH₂ₓ—O—, wherein x is 6.

In one embodiment of the process according to the present invention, a compound of formula (I.3) is obtained:

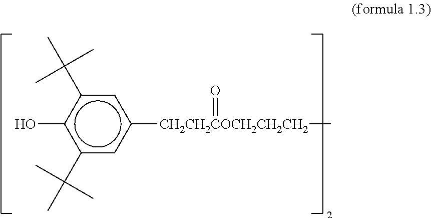

In one embodiment of the process according to the present invention, the integer of n in formula (I) or (II) is 2, and A of formula (I) or (III) has the formula O—CₓH₂ₓ—S—CₓH₂ₓ—O, wherein x is 2.

In one embodiment of the process according to the present invention, a compound of formula (I.4) is obtained:

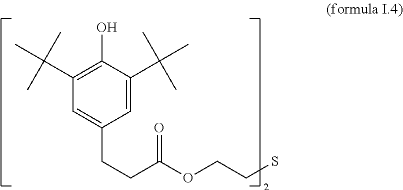

In one embodiment of the process according to the present invention, the integer of n in formula (I) or (II) is 2, and A of formula (I) or (III) has the formula —NH—CₓH₂ₓ—NH—, wherein x is 6.

In one embodiment of the process according to the present invention, a compound of formula (I.5) is obtained:

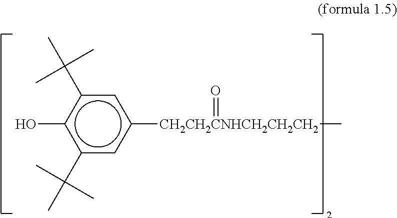

In one embodiment of the process according to the present invention, the integer of n in formula (I) or (II) is 2, and A of formula (I) or (III) has the formula —O—(CH₂CH₂O)ₐCH₂CH₂O—, wherein a is 2.

In one embodiment of the process according to the present invention, a compound of formula (I.6) is obtained:

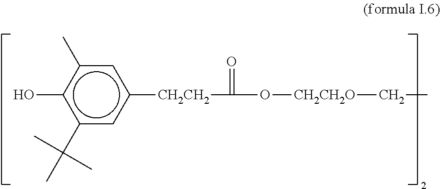

In one embodiment of the process according to the present invention, the integer of n in formula (I) or (II) is 2, and A of formula (I) or (III) has the formula —O—(CH₂CH₂O)ₐCH₂CH₂O—, wherein a is is 1 to 8.

In one embodiment of the process according to the present invention, the integer of n in formula (I) or (II) is 2, and A of formula (I) or (III) has the formula —O—(CH₂CH₂O)ₐCH₂CH₂O—, wherein a is on average 2.

In one embodiment of the process according to the present invention, a compound of formula (I.7) is obtained:

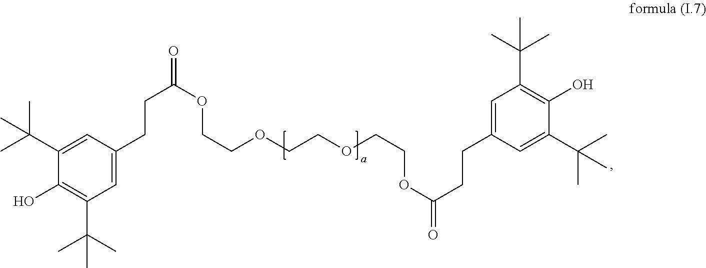

wherein a is 0 to 7.

In one embodiment of the process according to the present invention, a compound is obtained, wherein the unit

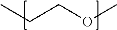

may not be present (a is 0) in the compound of formula (I.7).

In one embodiment of the process according to the present invention, a compound is obtained, wherein the unit

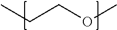

is repeated up to 7 times (a is 7) in the compound of formula (I.7).

In one embodiment of the process according to the present invention, the integer of n in formula (I) or (II) is 3.

In one embodiment of the process according to the present invention, the integer of n in formula (I) or (II) is 3, and A of formula (I) or (III) has the formula H₃C—CₓH₂ₓ—C(C_{y}H_{2y}O—)₃, —O—CₓH₂ₓCH₂(O—)—C_{y}H_{2y}—O—, wherein x and yare independently from one another an integer selected from 1 to 8.

In one embodiment of the process according to the present invention, the integer of n in formula (I) or (II) is 4.

In one embodiment of the process according to the present invention, the integer of n in formula (I) or (II) is 4 and A is the group —C(—CH₂—O—)₄.

In one embodiment of the process according to the present invention, a compound of formula (I.8) is obtained:

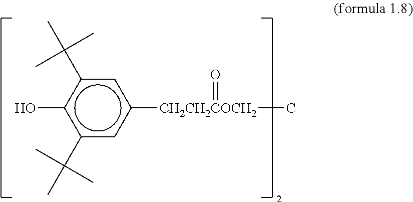

The compounds of formulae (II) and (III) used in the novel process to obtain compounds of formula (I) are known, and can be prepared by already known processes, and the compounds of formulae (I.1) to (I.8) known as such can be obtained by the new and inventive process according to the present invention.

The compounds of formula (I) obtained in the practice of this invention are used typically for protecting organic materials which are subject to thermal, oxidative and/or actinic degradation, including plastics materials and lubricants, and some are commercially available.

In comparison to the process in the prior art, the process of the present invention has several advantages:

At the end of the reaction, the catalyst, the alkali metal (bi) carbonate is dispersed as salt in the final product melt, and can just be filtered off, and does not require to be first deactivated by hydrolysis and precipitated beforehand.

In addition to the omitted washing step, the further addition of other reactive agents such as “washing agents” or “halide scavengers” are not required.

The residue of the remaining catalyst is very low (on average 10 ppm of the alkali metal at max) and has—especially in the case of sodium—no relevance with regard to toxicity and food contact regulations (in comparison, aluminum residues underlie prescriptive limits for food safety).

Particular attention is drawn to the fact that, in the process according to the invention, discolorations in the reaction mass and in the products are avoided. The discoloration problems referred to at the outset resulting from the catalyst are not encountered.

In general, the product obtained shows a long-term stability, which increases its shelf-life and leads to more sustainable solutions.

In case sodium bicarbonate is applied as catalyst in the transesterification, the transmission rate is even better than for potassium bicarbonate, as the latter shows lower values of the transmission, appearing visually “colored”. Hence, sodium bicarbonate, even if applied in higher dosages in the process according to the present invention, does not show any influence on the color.

The produced methanol can easily evaporate from the reaction mixture, being thereby removed from the equilibrium and leading to high conversion rate on the side of the desired product.

Any optionally remaining excess raw materials may be distilled off with standard methods or may alternatively be removed by recrystallization.

The novel process does not require any solvent. The process can be operated with only the two reactant and the catalyst being present. All components may be present in a solid state, meaning the two reactants and the catalyst. Alternatively, one reactant may be melted to a liquid state of matter, or both reactants may be liquid. The catalyst is either present from the beginning, meaning in a mixture with the one or both reactant(s), or may subsequently be added en bloc or portion wise to and dispersed in the solid, semi-liquid or liquid mixture of the reactants. The transesterification starts when the catalyst is added and the mixture is heated.

Preferably the reaction is free of water. Free of water means, that no additional water is added to reactant mixture. A certain humidity of the reactants due to optional hygroscopic properties can be tolerated (up to 0.1 weight % of water content). Preferably the reactant mixture is heated at the start of the reaction in order to remove the humidity of the chemical reactants.

Thus, the novel process does not require the presence of a solvent.

However, the novel process may also be carried out in the presence of a predominantly inert organic solvent, which may serve merely for dilution purposes.

Typically, the optional organic solvent is selected from an aliphatic or aromatic hydrocarbon such as pentane, hexane, heptane, octane, decaline, cyclohexane, benzene, toluene, xylene(s), mesitylene, dichloromethane, chloroform, tetrachloromethane, bromoform, petroleum ether, tetrahydrofuran or mixture thereof.

However, activating solvents such as dimethylformamide (DMF), dimethylacetamide (DMAc or DMA), N-methyl-2-pyrrolidone (NMP), hexamethylphosphoramide (HMPA) and Dimethyl sulfoxide (DMSO) are not suitable for a process according to the present invention. Their interaction with the reactants of the reaction mixture would lead to undesired side effects and side products. For instance, DMSO, one of the strongest organic solvents, is regarded as an unfavorable solvent in this context, as it dissolves inorganic salts and is laborious and difficult to remove from the reaction mixture and could lead to unwanted toxic residues.

The transesterification reaction can be carried out in the temperature range from 120 to 220° C. Preferably the transesterification reaction can be carried out in the temperature range from 130 to 200° C., more most preferably from 150 to 190° C.

The transesterification reaction can be operated at atmospheric pressure of 1000 mbar (specifically 1013 mbar) but is preferably operated at a lower range of 300 to 1 mbar. Preferably the transesterification reaction is operated in a range from 200 to 1 mbar, and more preferably from 100 to 1 mbar.

As methanol is formed during the reaction, the pressure can change in the course of the reaction. For example, the pressure rises commensurately with the amount of methanol formed. If the methanol is removed, then it is expedient to reduce the pressure until any excess of methanol is separated from component of formula (III).

The catalyst can be added to the reaction mixture of compound of formula (II) and compound of formula (III) in concentration of less than 10 wt %, preferably less than 5 wt %.

Conveniently the catalyst is added to the reaction mixture in a concentration range of 0.1 to 5 wt %, more preferably from 0.2 to 2 wt % and most preferably from 0.25 to 1 wt %.

Customary operations such as stirring the reaction mixture are useful.

Further, as already mentioned above, the transesterification reaction step of the present inventions takes 1 to 8 hours, preferably from 4 to 6 hours.

When the reaction is completed, the alkali metal (bi)carbonate, which is dispersed in the liquid mixture, may simply be removed by filtration. As mentioned further above, the filtration step is very simple and does neither require several filtration steps nor additional filtration aids.

Contrary to the catalyst discussed in the prior art, there is no need to destroy remaining concentrations by acidifying the reaction mass with an acid or to use washing agents. And due to its harmlessness and ubiquity with regard consumer safety and environmental concerns, no further precautious measures need to be taken. As already pointed out further above, methanol, which has been formed during the reaction, is easily removed by simple evaporation during the reaction at elevated temperature, and any remaining methanol is finally removed when vacuum is applied.

The product of formula (I) could optionally either be crystallized directly by cooling and inoculating the reaction melt, or by taking up the reaction melt in a suitable solvent, cooling the solution and effecting crystallization by inoculation. In such case, suitable solvents are hydrocarbons as mentioned above, such as pentane, hexane, heptane, octane, cyclohexane, decaline, petroleum ether or mixtures thereof; aromatic hydrocarbons such as benzene, toluene or xylene. However, due to simplicity of the process and the harmlessness of the catalyst, this additional step can and—for energy saving purposes—should be omitted, as no advantages are to be observed.

In general, about equivalent amounts of the ester (II) and the alcohol (III) are used, meaning a ratio of about 1:1 applied. However, a slight excess (of about 1 to 2 wt %) of the more volatile reactant may be present, which would to be removed by distillation afterwards.

Hence, depending on the volatility, the ratio of reactant (II) (or Ill) per equivalent of reactant (III) (or II) is conveniently from 0.8:1 to 1.3:1, preferably from 0.85:1 to 1.2:1, whereby ratio of the more volatile reactant is higher.

### EXAMPLES

### GC-Method:

The samples were analyzed by gas chromatography (GC), wherein the method detected individual components qualitatively from a sample depending on their individual retention times. The concentration of the individual component in the sample are given in it is percental peak area as gas chromatographic area percent [GC area %]. The assignment of the peaks was done by measuring the individual starting materials and the product as single component. The gas chromatograph from the company Agilent Technologies was equipped with an Optima 5 MS Accent (50 m×0.2 mm×0.35 μm, Macherey-Nagel) column. The injection temperature was 280° C. and the injection volume 0.2 μL. A split ratio of 1 to 50 was used. The carrier gas was hydrogen with a constant flow of 3.0 mL/min. The initial temperature was 50° C. with a holding time of 2 minutes. The following ramp was 25° C./min to 300° C. with a holding time of 19 minutes. The detection temperature was 320° C. and the total runtime was 31 minutes.

### HPLC-Method:

The samples were analyzed by high performance liquid chromatography (HPLC), wherein the method detected individual components qualitatively from a sample depending on their individual retention times. The quantification is done by HPLC area % method at wavelength 275 nm. The assignment of the peaks was done by measuring the individual starting materials and the product as single component.

The high performance liquid chromatograph from the company Agilent Technologies was equipped with an Halo C8 column (150×3.0 mm, AMT). The injection volume 3.0 μL. The flow rate was 1.0 mL/min. The temperature was 30° C. The instrument was run under gradient elution conditions. The mobile phase A was acetonitrile/water 70:30 (v/v). The mobile phase B was Acetonitrile/Methyl, t-butyl ether (MTBE) 70:30 (v/v). The gradient runs from 100% mobile Phase A to 100% mobile phase B in 12 minutes, holds mobile phase B for additional 3 minutes. The detection wavelength was 275 nm.

The invention is illustrated in more detail by the following non-limitative Examples in which parts and percentages are by weight, unless otherwise specified.

### Examples for NaHCO₃—Variation of Catalyst Loading and Pressure-Time Steps

### General Procedure for Example No 1-9

A vessel was charged with methyl 3-(3,5-di-tert-butyl-4-hydroxyphenyl)propionate (326 g, 1.12 mol) and stearyl alcohol (300 g, 1.10 mol) under a nitrogen flow and heated. Once the temperature of the reaction mixture has reached 130° C., vacuum (20 mbar) was applied. After 20 minutes the vessel was ventilated with nitrogen and the temperature was increased to 200° C. At an internal temperature of 190° C., NaHCO₃ (6.2 g for 1 wt %, 3.1 g for 0.5 wt %, 1.5 g for 0.25 wt %) was added, and the reaction was run under different pressure-time steps. The product yield is expressed as conversion rate in GC area % of the target molecule in obtained reaction mixture.

The different catalyst loadings and pressure-time steps are outlined in table 1.

TABLE 1
Variation of process parameters (catalyst
loading, pressure-time steps).

| Exam- | | Catalyst | | | Conversion |
|---|---|---|---|---|---|
| ple | | loading | | Pressure [mbar] | [GC |
| No | Catalyst | [wt %] | | (time) | area % |
| 1 | NaHCO₃ | 1 | wt % | 1013 (2 h)/100 (2 h)/ | 91.05 |
| | | | | 50 (2 h) | |
| 2 | NaHCO₃ | 1 | wt % | 1013 (1 h)/200 (1 h)/ | 90.33 |
| | | | | 100 (1 h)/50 (3 h) | |
| 3 | NaHCO₃ | 1 | wt % | 1013 (1 h)/200 (1 h)/ | 90.10 |
| | | | | 100 (1 h)/50 (1 h)/10 | |
| | | | | (2 h) | |
| 4 | NaHCO₃ | 1 | wt % | 200 (1 h)/100 (1 h)/10 | 93.48 |
| | | | | (4 h) | |
| 5 | NaHCO₃ | 0.5 | wt % | 1013 (2 h)/100 (2 h)/ | 90.55 |
| | | | | 50 (2 h) | |
| 6 | NaHCO₃ | 0.5 | wt % | 200 (1 h)/100 (1 h)/20 | 92.69 |
| | | | | (4 h) | |
| 7 | NaHCO₃ | 0.25 | wt % | 200 (1 h)/100 (1 h)/20 | 90.98 |
| | | | | (4 h) | |
| 8 | NaHCO₃ | 0.25 | wt % | 200 (2 h)/10 (4 h) | 90.22 |
| 9 | NaHCO₃ | 0.25 | wt % | 200 (1 h)/100 (1 h)/10 | 91.75 |
| | | | | (4 h) | |

All examples in table 1 show a very high conversion rate of more than 90 GC area %. For instance, for example 8 with a conversion rate of 90.22 GC area %, the loading of the catalyst was 0.25 weight % of the total loading in the vessel and the reaction was first run at a pressure of 200 mbar for two hours, and thereafter the pressure was reduced to 10 mbar for four hours, so that the total reaction time was six hours. In comparison, for example 4, with a conversion rate of 93.48 GC area %, the loading of the catalyst was one weight % of the total loading in the vessel and the reaction was first run at a pressure of 200 mbar for one hour, then the pressure was reduced to 100 mbar for another hour and thereafter the reaction was run at 10 mbar for four hours. The total reaction time in example 4 was six hours.

### Examples for Na₂CO₃

### General Procedure for Example No 10-11

A vessel was charged with methyl 3-(3,5-di-tert-butyl-4-hydroxyphenyl)propionate (326 g, 1.12 mol) and stearyl alcohol (300 g, 1.10 mol) under a nitrogen flow and heated. Once the internal temperature of the reaction mixture has reached 130° C., vacuum (20 mbar) was applied. After 20 minutes the vessel was ventilated with nitrogen and the temperature was increased to 200° C. At an internal temperature of 190° C., Na₂CO₃ (6.2 g, 58.5 mmol, 1 wt %) was added, and the reaction was run under different pressure-time steps. The product yield is expressed as conversion rate in GC area % of the target molecule in obtained reaction mixture.

The varied pressure-time steps are outlined in table 2.

TABLE 2
Variation of process parameters (pressure-time steps).

| Exam- | | Catalyst | | |
|---|---|---|---|---|
| ple | | loading | Pressure [mbar] | Conversion |
| No | Catalyst | [wt %] | (time) | [GC area %] |
| 10 | Na₂CO₃ | 1 wt % | 1013 (2 h)/100 (2 h)/50 (2 h) | 82.62 |
| 11 | Na₂CO₃ | 1 wt % | 200 (1 h)/100 (1 h)/20 (4 h) | 89.69 |

Both examples in table 2 show a high conversion rate of more the 80 GC area %. It can be seen that within the same time frame of 6 hours, the conversion rate can be improved by decreasing the pressure to 200 mbar and below from the beginning (example 11), compared to a transesterification process (example 10), wherein the pressure is kept at room temperature of 1013 mbar during one third of the time (2 hours).

### Example for Variations in Catalyst Addition

### Procedure for Example No 12

A vessel was charged with methyl 3-(3,5-di-tert-butyl-4-hydroxyphenyl)propionate (326 g, 1.12 mol) and stearyl alcohol (300 g, 1.10 mol) under a nitrogen flow and heated. Once the internal temperature of the reaction mixture has reached 130° C., vacuum (20 mbar) was applied. After 20 minutes the vessel was ventilated with nitrogen and the temperature was increased to 200° C. At an internal temperature of 190° C., NaHCO₃ (3.10 g, 0.5 wt %) was added. The reaction was first run at atmospheric pressure for two hours. Thereafter another 0.5 wt % of NaHCO₃ (3.10 g) were added and the pressure was reduced to 100 mbar for two hours and at last the reaction was run at 50 mbar for two hours.

The product was obtained with 91.04% in yield, which expressed as conversion rate in GC area % of the target molecule in obtained reaction mixture.

TABLE 3

| | | 1^{st} catalyst | Pressure | 2^{nd} catalyst | Pressure | |
|---|---|---|---|---|---|---|
| Example | | loading | [mbar] | loading | [mbar] | Conversion |
| No | Catalyst | [wt %] | (time) | [wt %] | (time) | [GC area %] |
| 12 | NaHCO₃ | 0.5 wt % | 1013 (2 h) | 0.5 | 100 (2 h)/ | 91.04 |
| | | | | | 50 (2 h) | |
| 1 | NaHCO₃ | 1 wt % | 1013 (2 h)/ | / | / | 91.05 |
| | | | 100 (2 h)/ | | | |
| | | | 50 (2 h) | | | |

In comparison to example 1, wherein the total 1 weight % of NaHCO₃ was added only once at beginning of the reaction, and the reaction was further run under the same different pressure-time steps, no significant change of conversion rate was observed.

### Examples for Variations of Different Educts

### Example No. 23: Synthesis of Poly(oxy-1,2-ethanediyl)-alpha-[3-[3,5-bis(1,1-dimethylethyl)-4-hydroxyphenyl]-1-oxopropyl]-omega-[3-[3,5-bis(1,1-dimethylethyl)-4-hydroxyphenyl]-1-oxopropoxy] [CAS-No. 36541-61-4]

A vessel was charged with methyl 3-(3,5-di-tert-butyl-4-hydroxyphenyl)propionate (340 g, 1.16 mol), PEG200 (97.0 g, 485 mmol) and NaHCO₃ (1.10 g, 0.25 wt %) under a nitrogen flow and heated at ambient pressure. Once the internal temperature of the reaction mixture had reached 140° C., a vacuum of 200 mbar was applied. After one hour reaction time the pressure started to be gradually reduced to eventually retain 20 mbar after a total reaction time of two hours. After three hours reaction time the temperature was gradually increased to 190° C. These conditions (temperature of 190° C. and pressure of 20 mbar) were kept then kept for further six hours of reaction time. The final product was obtained with 98% in yield, which expressed as conversion rate in GC area % of the target molecule in obtained reaction mixture.

### Example No. 24: Synthesis of Tetrakis[3-(4-hydroxy-3,5-di-tert-butylphenyl) propionyloxymethyl]methane [CAS-No. 6683-19-8]

A vessel was charged with methyl 3-(3,5-di-tert-butyl-4-hydroxyphenyl)propionate (341 g, 1.16 mol), pentaerythritol (44.6 g, 327 mmol) and NaHCO₃ (1.45 g, 0.25 wt %) under a nitrogen flow and heated at ambient pressure. After having reached an internal temperature of 190° C., the pressure was stepwise reduced from 1013 to 20 mbar within one hour, and the reaction mixture was kept for further five hours under said conditions (temperature of 190° C. and pressure of 20 mbar).

The final product was obtained with 76% in yield, which expressed as conversion rate in HPLC area % of the target molecule in obtained reaction mixture.

### Example No. 25: Synthesis of 2-Ethylhexyl 3-(3,5-di-tert-butyl-4-hydroxyphenyl) propanoate 2,2′-Methylenebis(6-tert-butyl-4-methylphenol) [CAS-No. 144429-84-5]

A vessel was charged with methyl 3-(3,5-di-tert-butyl-4-hydroxyphenyl)propionate (341 g, 1.16 mol) and 2-ethyl hexanol (173 g, 1.33 mol) under a nitrogen flow and heated at ambient pressure. Once the internal temperature of the reaction mixture had reached 160° C., NaHCO₃ (1.28 g, 0.25 wt %) was added and subsequently the pressure was stepwise reduced from 1013 to 200 mbar and kept for one hour. Then the temperature was stepwise increased to reach 180° C. whereby the pressure was reduced to finally reach 80 mbar. The total reaction time after the addition of NaHCO₃ was six hours.

The product was obtained with 89% in yield, which expressed as conversion rate in GC area % of the target molecule in obtained reaction mixture.

### Example No. 26: Synthesis of Ethylenebis(oxyethylene)bis[3-(5-tert-butyl-4-hydroxy-m-tolyl) propionate] [CAS-No. 36443-68-2]

A vessel was charged with methyl 3-(3-tert-butyl-4-hydroxy-5-methylphenyl)propionate (431 g, 1.72 mol), triethylene glycol (128 g, 852 mmol) and NaHCO₃ (1.40 g, 0.25 wt %) under a nitrogen flow and heated at ambient pressure. Once the internal temperature of the reaction mixture had reached 120° C. the pressure was reduced from 1013 to 200 mbar. Within one hour the temperature was increased to reach 180° C. while keeping the pressure. Thereafter the temperature was kept at 180° C. while reducing the pressure to finally reach 20 mbar over a time period of two hours and further kept for eight hours under said conditions. The product was obtained with 89% in yield, which expressed as weight % of the target molecule in obtained reaction mixture.

### Examples for Transmission Measurements

### General Procedure for Example No 13-14-22

A vessel was charged with methyl 3-(3,5-di-tert-butyl-4-hydroxyphenyl)propionate (326 g, 1.12 mol) and stearyl alcohol (300 g, 1.10 mol) under a nitrogen flow and heated. Once the internal temperature of the reaction mixture has reached 130° C., vacuum (20 mbar) was applied. After 20 minutes the vessel was ventilated with nitrogen and the temperature was increased to 200° C. At an internal temperature of 190° C., the catalyst (6.2 g, 58.5 mmol, 1 wt %) was added. The reaction was first run at atmospheric pressure for two hours, thereafter the pressure was reduced to 100 mbar for two hours and at last the reaction was run at 50 mbar for two hours. The mixture was released from the vessel under a nitrogen flow and filtered under inert conditions. Thereafter the transmission of the sample was measured in toluene (c=0.1 g/mL) at 425 nm and 500 nm.

The results of the transmission measurements are listed in table 4.

TABLE 4
Results of transmission measurements.

| Example | | Transmission at | Transmission at |
|---|---|---|---|
| No | Catalyst | 425 nm [%] | 500 nm [%] |
| 13 | NaHCO₃ | >99.9 | >99.9 |
| 14 | KHCO₃ | 93.18 | 97.92 |
| 22 | Na₂CO₃ | 98.7 | >99.9 |

The values in table 4 reflect, that whereas the transmission rate of 99.9% for the use of NaHCO₃ and Na₂CO₃ as catalyst demonstrate almost full transmission by being “colorless” or achromatic, the transmission rate of KHCO₃, although still being above 90% at both wavelengths, is slightly below the one of NaHCO₃, and appears to show visually a slightly colored with a yellowish-brownish undertone. Similarly, and comparable to KHCO₃, K₂CO₃ shows a slightly yellowish undertone color.

### Comparative Examples with Other Catalysts

### General Procedure for Example No 15-20

A vessel was charged with methyl 3-(3,5-di-tert-butyl-4-hydroxyphenyl)propionate (326 g, 1.12 mol) and stearyl alcohol (300 g, 1.10 mol) under a nitrogen flow and heated. Once the internal temperature of the reaction mixture has reached 130° C., vacuum (20 mbar) was applied. After 20 minutes the vessel was ventilated with nitrogen and the temperature was increased to 200° C. At an internal temperature of 190° C., the catalyst (6.2 g, 58.5 mmol, 1 wt %) was added. The reaction was first run at atmospheric pressure for two hours, thereafter the pressure was reduced to 100 mbar for two hours and at last the reaction was run at 50 mbar for two hours. The product yield is expressed as conversion in GC area % of the target molecule in obtained reaction mixture.

The different catalysts used are listed in Table 5.

TABLE 5
Variation in catalyst.

| Example | | Conversion [GC |
|---|---|---|
| No | Catalyst | area %] |
| 10 | Na₂CO₃ | 82.62 |
| 15 | Al(OⁱPr)₃ | 93.67 |
| 16 | NaOAc | 71.36 |
| 17 | KOAc | 71.49 |
| 18 | Li₂CO₃ | 48.62 |
| 19 | K₂CO₃ | 89.69 |
| 20 | KHCO₃ | 93.20 |
| 1 | NaHCO₃ | 91.05 |

From the data shown in table 5, it can be seen that alkali metal bicarbonate or an alkali metal carbonate can be an alternative to other catalysts used for the transesterification, and that especially the alkali metal bicarbonates may have a comparable efficacy as the aluminum catalysts but without their disadvantages as discussed further above.

### Comparative Examples with Regard to Prior Art (SU1014213)

### General Procedure for Example No 21

A vessel was charged with methyl 3-(3,5-di-tert-butyl-4-hydroxyphenyl)propionate (261 g, 891 mmol) and stearyl alcohol (240 g, 883 mmol) under a nitrogen flow and heated. Once the internal temperature of the reaction mixture has reached 130° C., vacuum (20 mbar) was applied. After 20 minutes the vessel was ventilated with nitrogen and at an internal temperature of 140° C. the catalyst mixture of K₂CO₃, Na₂CO₃ and phenol (K₂CO₃: 3.90 g; Na₂CO₃: 7.80 g; phenol 11.9 g, in total: 4.71 wt %) was added. The reaction was first run at 200 mbar for 0.5 hour, thereafter the pressure was reduced to 100 mbar for 0.5 hours and at last the reaction was run at 80 mbar for eight hours.

Finally, after nine hours of reaction time, the conversion (expressed in GC area %) of the target molecule in the obtained mixture was: 88.88%.

The obtained product yield was analyzed by gas chromatography (GC), wherein the individual desired components were detected dependent on their individual retention times. Their concentration was determined in their respective percental peak area as GC area %.

It was found that a total of 2.28% of the conversion (expressed in GC area %) consisted of several detected unknown side products being formed during the comparative transesterification reaction, whereas 8.84% of non-converted educts were detected as well.

For comparison, in example 4 according to the present invention, the use of solely 1 wt % of NaHCO₃ as catalyst in the transesterification process according to the present invention, resulted in a total amount of only 0.34 GC area % undesired compounds detected by GC, which were found to be merely impurities of educts.

In addition to the comparatively high amount of side products obtained according to the process in the prior art with the ternary catalyst mixture, the transmission results were as well disadvantageous.

The transmission of the samples was measured in toluene (c=0.1 g/mL) at 425 nm and 500 nm. The results of the transmission measurements are listed below in table 6.

TABLE 6
Results of transmission measurements in comparison.

| Example | | Transmission at | Transmission at |
|---|---|---|---|
| No | Catalyst | 425 nm [%] | 500 nm [%] |
| 13 | NaHCO₃ | >99.9 | >99.9 |
| 14 | KHCO₃ | 93.18 | 97.92 |
| 21 | K₂CO₃ + | 87.20 | 96.59 |
| | Na₂CO₃ + | | |
| | Phenol | | |

1.-16. (canceled)

17. A non-aqueous process for the preparation of a compound of formula (I) 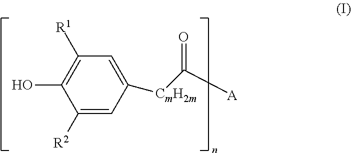
wherein
R₁ and R₂ are independently of one another hydrogen or a linear or branched C₁-C₈-alkyl,
m is either 0 or an integer selected from 1, 2 or 3,
n is an integer selected from 1, 2, 3 or 4,
and
A is
if n is 1, OR₃, and R₃ is C₄-C₂₀-alkyl or C₅-C₁₂ cycloalkyl, or
if n is 2, has the formula —O—CₓH₂ₓ—O—, O—CₓH₂ₓ—S—CₓH₂ₓ—O, —NH—CₓH₂ₓ—NH— or —O—(CH₂CH₂O)ₐCH₂CH₂O—,
wherein
x is an integer selected from 2 to 8, and
a is an integer selected from 1 to 12,
if n is 3, has the formula H₃C—CₓH₂ₓ—C(C_{y}H_{2y}O—)₃, —O—CₓH₂ₓ—CH₂(O—)—C_{y}H_{2y}—O—,
wherein
x,y are independently from one another an integer selected from 1 to 8;
if n is 4, has the formula —C(—CH₂—O—)₄
by a transesterification reaction, wherein a compound of formula (II) 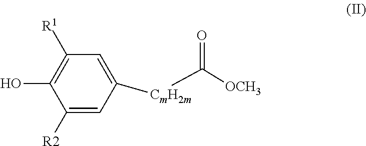
is reacted with a compound of formula (III) 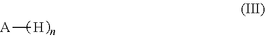
and wherein the process step of the transesterification reaction between compound of formula (II) and compound of formula (III) is characterized by being started by the addition of catalyst consisting of an alkali metal bicarbonate or an alkali metal carbonate selected from the group consisting of NaHCO₃, Na₂CO₃, KHCO₃ und K₂CO₃, and which is present in its undissolved solid form.

18. A process according to claim 17, wherein m is 2.

19. A process according to claim 17, wherein R₁ and R₂ are independently from one another a linear or branched C₁-C₄ alkyl.

20. A process according to claim 17, wherein R₁ and R₂ are both tert-butyl.

21. A process according to claim 17, wherein n is 1 and R₃ is C₈-C₁₈ alkyl.

22. A process according to claim 17, wherein n is 4 and A is the group —C(—CH₂—O)₄.

23. A process according to claim 17, which is conducted in the absence of a solvent.

24. A process according to claim 17, wherein the catalyst is NaHCO₃ or Na₂CO₃.

25. A process according to claim 24, wherein the catalyst is NaHCO₃.

26. A process according to claim 17, wherein the reaction is carried out in the temperature range from 120 to 220° C.

27. A process according to claim 17, wherein the process is operated at atmospheric pressure of 1000 mbar or lower.

28. A process according to claim 27, wherein the process is operated in a range of 300 to 1 mbar.

29. A process according to claim 17, wherein after the reaction the catalyst is removed from the obtained product by filtration.

30. A process according to claim 17, wherein the catalyst is added to the mixture of compound of formula (II) and compound of formula (III) in concentration of less than 5 wt %.

31. A process according to claim 17, wherein the transesterification process step is allowed to take place for 1 to 8 hours.

32. A process according to claim 17, which is in the presence of an organic inert solvent selected from pentane, hexane, heptane, octane, decaline, cyclohexane, benzene, toluene, xylene(s), mesitylene, dichloromethane, chloroform, tetrachloromethane, bromoform, petroleum ether, tetrahydrofuran or mixture thereof.
